(19) United States
(12) Patent Application Publication
(10) Pub. No.: US 2026/0000453 A1
(43) Pub. Date: Jan. 1, 2026

(54) INTRAVASCULAR LITHOTRIPSY BALLOON

(71) Applicant: Bolt Medical, Inc., Carlsbad, CA (US)

(72) Inventors: Eric Schultheis, San Clemente, CA (US); Alan Duong, San Diego, CA (US); Thomas E. Waggoner, Tucson, AZ (US); Christopher A. Cook, Laguna Niguel, CA (US)

(73) Assignee: Bolt Medical, Inc., Carlsbad, CA (US)

(21) Appl. No.: 19/320,226

(22) Filed: Sep. 5, 2025

Related U.S. Application Data
(63) Continuation of application No. 17/707,696, filed on Mar. 29, 2022.
(60) Provisional application No. 63/171,213, filed on Apr. 6, 2021.

(51) Int. Cl. A61B 18/26; A61B 18/00; A61M 25/10

(52) U.S. Cl. CPC A61B 18/26; A61B 2018/00285; A61M 25/1002

(57) ABSTRACT

A catheter system (100) for treating a treatment site (106) within or adjacent to a vessel wall (108) or heart valve includes a balloon (104) having a balloon length (204L). The balloon (104) is configured to be movable between an inflated state (135) and a deflated state. The balloon (104) includes (i) a balloon proximal region (250), (ii) a balloon distal region (254), and (iii) a balloon transition region (252). The balloon proximal region (250) has a balloon proximal end (204P) and a substantially constant balloon proximal region diameter (256) while the balloon (104) is in the inflated state (135). The balloon distal region (254) has a balloon distal end (104D), and a substantially constant balloon distal region diameter (262) while the balloon (104) is in the inflated state (135). The balloon distal region diameter (262) is different than the balloon proximal region diameter (256). The balloon transition region (252) has a balloon transition region diameter (260) that varies. The balloon transition region (252) is positioned between the balloon proximal region (250) and the balloon distal region (254).

### RELATED APPLICATION

This application is a continuation of and claims the benefit of the earlier filing date of U.S. patent application Ser. No. 17/707,696, filed Mar. 29, 2022, which claims the benefit of priority under 35 U.S.C. § 119 to U.S. Provisional Application No. 63/171,213, filed on Apr. 6, 2021, and which applications are incorporated herein by reference in their entireties for all purposes. Any and all priority claims identified in the Application Data Sheet, or correction thereto, are hereby incorporated by reference under 37 C.F.R. § 1.57.

### BACKGROUND

Vascular lesions within and adjacent to vessels in the body can be associated with an increased risk for major adverse events, such as myocardial infarction, embolism, deep vein thrombosis, stroke, and the like. Severe vascular lesions can be difficult to treat and achieve patency for a physician in a clinical setting.

Vascular lesions may be treated using interventions such as drug therapy, balloon angioplasty, atherectomy, stent placement, vascular graft bypass, to name a few. Such interventions may not always be ideal or may require subsequent treatment to address the lesion.

### SUMMARY

The present invention is directed toward a catheter system for treating a treatment site within or adjacent to a vessel wall or heart valve. The catheter system includes an inflatable balloon. In various embodiments, the inflatable balloon can be configured to be movable between an inflated state and a deflated state. The inflatable balloon can include a balloon proximal region having a proximal region diameter in the inflated state and a balloon distal region having a distal region diameter in the inflated state that is different than the proximal region diameter in the inflated state. The inflatable balloon can include at least one balloon transition region having a balloon transition region diameter that varies in the inflated state. The balloon transition region can be positioned between the balloon proximal region and the balloon distal region.

In certain embodiments, the inflatable balloon is formed from a non-compliant material.

In some embodiments, the inflatable balloon is formed from a semi-compliant material.

In various embodiments, the inflatable balloon has a balloon length of greater than 1 millimeter and less than 300 millimeters.

In certain embodiments, the catheter system further includes an energy carrier having (i) a carrier proximal end and (ii) a carrier distal end positioned within the inflatable balloon.

In some embodiments, the energy carrier is an optical fiber.

In various embodiments, the catheter system further includes an energy source in optical communication with the carrier proximal end.

In certain embodiments, the energy source is a laser.

The present invention is also directed toward a catheter system for treating a treatment site within or adjacent to a vessel wall or heart valve. The catheter system includes an inflatable balloon. The inflatable balloon can have a balloon length. The inflatable balloon can be configured to be movable between an inflated state and a deflated state. The inflatable balloon can include a balloon proximal region having a balloon proximal end. The balloon proximal region can have a balloon proximal region diameter while the inflatable balloon is in the inflated state. The balloon proximal region diameter can have a substantially constant diameter across the balloon proximal region. The inflatable balloon can include a balloon distal region having a balloon distal end, the balloon distal region having a balloon distal region diameter while the inflatable balloon is in the inflated state, the balloon distal region diameter having a substantially constant diameter across the balloon distal region, the balloon distal region diameter being different than the balloon proximal region diameter in the inflated state. The inflatable balloon can include a balloon transition region having a balloon transition region diameter that varies while the inflatable balloon is in the inflated state, the balloon transition region being positioned between the balloon proximal region and the balloon distal region.

In some embodiments, the balloon transition region has a transition region length that is greater than 1% and less than 99% of the balloon length.

In various embodiments, the balloon proximal region has a proximal region length is greater than 1% and less than 99% of the balloon length.

In certain embodiments, the balloon distal region has a distal region length that is greater than 1% and less than 99% of the balloon length.

In some embodiments, the balloon transition region diameter gradually decreases from the balloon proximal region to the balloon distal region.

In various embodiments, the balloon transition region includes a transition region transition portion.

In certain embodiments, the balloon transition region includes a plurality of transition region transition portions.

In some embodiments, each of the transition region transition portions are one of curved and tapered.

In various embodiments, the balloon transition region includes a plurality of transition region diameters each having a corresponding transition region linear portion.

In certain embodiments, the catheter system further includes an energy carrier having (i) a carrier proximal end and (ii) a carrier distal end positioned within the inflatable balloon.

In some embodiments, the energy carrier is an optical fiber.

In various embodiments, the optical fiber includes an emitter.

In certain embodiments, the optical fiber includes a pair of emitters.

In some embodiments, the balloon proximal region diameter is greater than 10% and less than 10000% of the balloon distal region diameter.

In various embodiments, the balloon transition region has an infinite number of balloon transition region diameters.

In certain embodiments, the balloon transition region has a plurality of balloon transition region diameters.

In some embodiments, the balloon transition region includes a first transition portion that gradually increases in diameter from the proximal end of the first transition portion to the distal end of the first transition portion.

In various embodiments, the balloon transition region includes a second transition portion that gradually decreases in diameter from the proximal end of the second transition portion to the distal end of the second transition portion.

In certain embodiments, the balloon transition region includes a third transition portion that gradually decreases in diameter from the proximal end of the third transition portion to the distal end of the third transition portion.

In some embodiments, the balloon transition region is angled relative to the balloon proximal region.

In various embodiments, the balloon transition region is angled relative to the balloon distal region.

In certain embodiments, the balloon proximal region has a substantially constant diameter.

In some embodiments, the balloon distal region has a substantially constant diameter.

In various embodiments, the balloon transition region has a plurality of diameters that are each different from one another.

In certain embodiments, the inflatable balloon includes a balloon wall having (i) a first wall thickness, and (ii) a second wall thickness that is different than the first wall thickness.

In some embodiments, the inflatable balloon includes a balloon wall having an infinite number of wall thicknesses.

In various embodiments, the balloon length is greater than 1 millimeter and less than 300 millimeters.

In certain embodiments, the catheter system further includes a light carrier having (i) a light carrier proximal end and (ii) a light carrier distal end positioned within the inflatable balloon.

In some embodiments, the light carrier is an optical fiber.

In various embodiments, the catheter system further includes an energy source in optical communication with the carrier proximal end.

In certain embodiments, the energy source is a laser.

The present invention is also directed to a method for manufacturing the catheter system of any of claims 9-39.

The present invention is also directed to a method for treating a treatment site within or adjacent to a vessel wall or heart valve. In various embodiments, the method can include the step of configuring an inflatable balloon to have a balloon length, the inflatable balloon being configured to be movable between an inflated state and a deflated state, the inflatable balloon including (i) a balloon proximal region having a balloon proximal end, the balloon proximal region having a balloon proximal region diameter while the inflatable balloon is in the inflated state, the balloon proximal region diameter having a substantially constant diameter across the balloon proximal region, (ii) a balloon distal region having a balloon distal end, the balloon distal region having a balloon distal region diameter while the inflatable balloon is in the inflated state, the balloon distal region diameter having a substantially constant diameter across the balloon distal region and (iii) a balloon transition region having a balloon transition region diameter that varies while the inflatable balloon is in the inflated state, the balloon transition region being positioned between the balloon proximal region and the balloon distal region.

The present invention is also directed toward a catheter system for treating a treatment site within or adjacent to a vessel wall or heart valve. The catheter system includes an inflatable balloon. The inflatable balloon can have a balloon length. The inflatable balloon can be configured to be movable between an inflated state and a deflated state, the inflatable balloon including (i) a balloon proximal region having a balloon proximal end, the balloon proximal region having a balloon proximal region diameter while the inflatable balloon is in the inflated state, the balloon proximal region diameter having a substantially constant diameter across the balloon proximal region, (ii) a balloon distal region having a balloon distal end, the balloon distal region having a balloon distal region diameter while the inflatable balloon is in the inflated state, the balloon distal region diameter having a substantially constant diameter across the balloon distal region, and (iii) a balloon transition region having a balloon transition region diameter that varies while the inflatable balloon is in the inflated state, the balloon transition region being positioned between the balloon proximal region and the balloon distal region, the balloon transition region extending from the balloon proximal region to the balloon distal region.

The present invention is also directed to a method for treating a treatment site within or adjacent to a vessel wall or heart valve. In various embodiments, the method can include the step of configuring an inflatable balloon to have a balloon length, the inflatable balloon being configured to be movable between an inflated state and a deflated state, the inflatable balloon including (i) a balloon proximal region having a balloon proximal end, the balloon proximal region having a balloon proximal region diameter while the inflatable balloon is in the inflated state, the balloon proximal region diameter having a substantially constant diameter across the balloon proximal region, (ii) a balloon distal region having a balloon distal end, the balloon distal region having a balloon distal region diameter while the inflatable balloon is in the inflated state, the balloon distal region diameter having a substantially constant diameter across the balloon distal region and (iii) a balloon transition region having a balloon transition region diameter that varies while the inflatable balloon is in the inflated state, the balloon transition region being positioned between the balloon proximal region and the balloon distal region.

The present invention is also directed toward a catheter system for treating a treatment site within or adjacent to a vessel wall or heart valve. The catheter system includes an inflatable balloon. The inflatable balloon can be configured to be movable between an inflated state and a deflated state, the inflatable balloon including (i) a balloon proximal region having a balloon proximal end, the balloon proximal region having a balloon proximal region diameter while the inflatable balloon is in the inflated state, the balloon proximal region diameter having a substantially constant diameter across the balloon proximal region, (ii) a balloon distal region having a balloon distal end, the balloon distal region having a balloon distal region diameter while the inflatable balloon is in the inflated state, the balloon distal region diameter having a substantially constant diameter across the balloon distal region, and (iii) a balloon transition region having a balloon transition region diameter that varies while the inflatable balloon is in the inflated state, the balloon transition region being positioned between the balloon proximal region and the balloon distal region, the inflatable balloon having a ratio of the proximal region length to the distal region length that is between 1:100 and 100:1.

The present invention is also directed toward a catheter system for treating a treatment site within or adjacent to a vessel wall or heart valve. The catheter system includes an inflatable balloon. The inflatable balloon can be configured to be movable between an inflated state and a deflated state, the inflatable balloon including (i) a balloon proximal region having a balloon proximal end, the balloon proximal region having a balloon proximal region diameter while the inflatable balloon is in the inflated state, the balloon proximal region diameter having a substantially constant diameter across the balloon proximal region, (ii) a balloon distal region having a balloon distal end, the balloon distal region having a balloon distal region diameter while the inflatable balloon is in the inflated state, the balloon distal region diameter having a substantially constant diameter across the balloon distal region, and (iii) a balloon transition region having a balloon transition region diameter that varies while the inflatable balloon is in the inflated state, the balloon transition region being positioned between the balloon proximal region and the balloon distal region, the inflatable balloon having a ratio of the proximal region diameter to the distal region diameter that is between 1:100 and 100:1.

The present invention is also directed toward a catheter system for treating a treatment site within or adjacent to a vessel wall or heart valve. The catheter system includes an inflatable balloon. The inflatable balloon can be configured to be movable between an inflated state and a deflated state. In various embodiments, the inflatable balloon can include (i) a balloon proximal region having a balloon proximal end, the balloon proximal region having a balloon proximal region diameter while the inflatable balloon is in the inflated state, the balloon proximal region diameter having a substantially constant diameter across the balloon proximal region, (ii) a balloon distal region having a balloon distal end, the balloon distal region having a balloon distal region diameter while the inflatable balloon is in the inflated state, the balloon distal region diameter having a substantially constant diameter across the balloon distal region and (iii) a balloon transition region having a balloon transition region diameter that varies while the inflatable balloon is in the inflated state, the balloon transition region being positioned between the balloon proximal region and the balloon distal region.

In some embodiments, the balloon transition region is linear.

In certain embodiments, the balloon transition region is curved.

The present invention is also directed toward a catheter system for treating a treatment site within or adjacent to a vessel wall or heart valve. The catheter system includes an inflatable balloon. The inflatable balloon can have a balloon length. The inflatable balloon can be configured to be movable between an inflated state and a deflated state, the inflatable balloon can include a balloon transition region having an infinite number of balloon transition region diameters that vary while the inflatable balloon is in the inflated state, the balloon transition region can extend substantially along the entire balloon length.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified schematic cross-sectional view of one embodiment of a catheter system having features of the present invention;

FIG. 2 is a partially transparent front view of one embodiment of a portion of the catheter system, including an optical fiber;

FIG. 3 is a partially transparent perspective view of the embodiment of a portion of the catheter system shown in FIG. 2, including the optical fiber;

FIG. 4 is a partially transparent front view of one embodiment of a portion of the catheter system, including an optical fiber;

FIG. 5 is a partially transparent front view of one embodiment of a portion of the catheter system, including an optical fiber;

FIG. 6 is a partially transparent front view of one embodiment of a portion of the catheter system, including an optical fiber;

FIG. 7A is a simplified cross-sectional view of one embodiment of a portion of the catheter system taken on line 7A-7A in FIG. 4; and

FIG. 7B is a simplified cross-sectional view of one embodiment of a portion of the catheter system taken on line 7B-7B in FIG. 4.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DESCRIPTION

Treatment of vascular lesions can reduce major adverse events or death in affected subjects. A major adverse event is one that can occur anywhere within the body due to the presence of a vascular lesion (also sometimes referred to herein as a “treatment site.” Major adverse events can include, but are not limited to, major adverse cardiac events, major adverse events in the peripheral or central vasculature, major adverse events in the brain, major adverse events in the musculature, or major adverse events in any of the internal organs.

As used herein, the treatment site can include a vascular lesion such as a calcified vascular lesion or a fibrous vascular lesion (hereinafter sometimes referred to simply as a “lesion”), typically found in a blood vessel or heart valve. Plasma formation can initiate a pressure wave and can initiate the rapid formation of one or more bubbles that can rapidly expand to a maximum size and then dissipate through a cavitation event that can also launch a pressure wave upon collapse. The rapid expansion of the plasma-induced bubbles can generate one or more pressure waves within a balloon fluid and thereby impart pressure waves upon the treatment site. The pressure waves can transfer mechanical energy through an incompressible balloon fluid to a treatment site to impart a fracture force on the lesion. Without wishing to be bound by any particular theory, it is believed that the rapid change in balloon fluid momentum upon a balloon wall of the inflatable balloon that is in contact with or positioned near the lesion is transferred to the lesion to induce fractures in the lesion.

Those of ordinary skill in the art will realize that the following detailed description of the present invention is illustrative only and is not intended to be in any way limiting. Other embodiments of the present invention will readily suggest themselves to such skilled persons having the benefit of this disclosure. Additionally, other methods of delivering energy to the lesion can be utilized, including, but not limited to, electric current induced plasma generation. Reference will now be made in detail to implementations of the present invention as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is appreciated that such a development effort might be complex and time-consuming, but would nevertheless be a routine undertaking of engineering for those of ordinary skill in the art having the benefit of this disclosure.

As used herein, the terms “intravascular lesion,” “vascular lesion,” and “treatment site” can be used interchangeably unless otherwise noted, and can include lesions located at or near blood vessels.

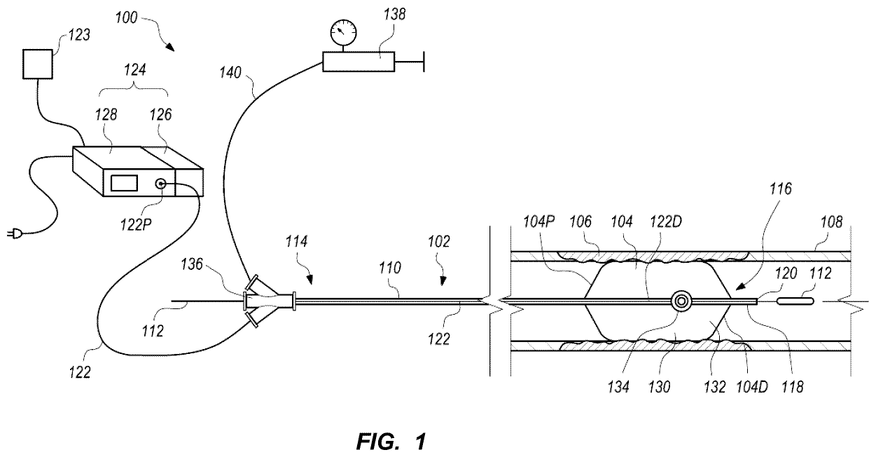

It is appreciated that the catheter systems herein can include many different forms and/or configurations other than those specifically shown and/or described herein. Referring now to FIG. 1, a simplified schematic cross-sectional view is shown of a catheter system in accordance with various embodiments herein. A catheter system 100 is suitable for imparting pressure to induce fractures in a vascular lesion within or adjacent to a vessel wall of a blood vessel or a heart valve. As used herein, the terms “vessel wall” and “heart valve” can be used interchangeably. In the embodiment illustrated in FIG. 1, the catheter system 100 can include one or more of a catheter 102, one or more optical fibers 122, a controller 123, a laser 124, a manifold 136, and a fluid pump 138.

The catheter 102 includes an inflatable balloon 104 (sometimes referred to herein simply as a “balloon”). The catheter 102 is configured to move to a treatment site 106 within or adjacent to a blood vessel 108. The treatment site 106 can include a vascular lesion such as a calcified vascular lesion, for example. Additionally, or in the alternative, the treatment site 106 can include a vascular lesion such as a fibrous vascular lesion.

The catheter 102 can include the balloon 104, a catheter shaft 110, and a guidewire 112. The balloon can be coupled to the catheter shaft 110. The balloon can include a balloon proximal end 104P and a balloon distal end 104D. The catheter shaft 110 can extend between a shaft proximal end 114 and a shaft distal end 116. The catheter shaft 110 can include a guidewire lumen 118 which is configured to move over the guidewire 112. The catheter shaft 110 can also include an inflation lumen (not shown). In some embodiments, the catheter 102 can have a distal end opening 120 and can accommodate and be moved over and/or along the guidewire 112 so that the balloon 104 is positioned at or near the treatment site 106.

The balloon 104 can include a balloon wall 130. The balloon 104 can expand from a collapsed configuration suitable for advancing at least a portion of the catheter shaft 102 through a patient's vasculature to an expanded configuration suitable for anchoring the catheter 102 into position relative to the treatment site 106.

The catheter shaft 110 of the catheter 102 can encircle one or more optical fibers 122 (only one optical fiber 122 is illustrated in FIG. 1 for clarity) in optical communication with a laser 124. The optical fiber 122 can be at least partially disposed along and/or within the catheter shaft 110 and at least partially within the balloon 104. In some embodiments, the catheter shaft 110 can encircle multiple optical fibers 122, such as a second optical fiber, a third optical fiber, etc.

The optical fiber 122 has a fiber proximal end 122P that is positioned at or adjacent to the laser 124. The optical fiber 122 extends between the laser 124 and the balloon 104. The optical fiber 122 is in optical communication with the laser 124.

The controller 123 can control the laser 124 so that the laser 124 can generate one or more energy pulses (e.g., the plasma pulse 134) as provided in greater detail herein. The controller 123 may also perform other relevant functions to control the operation of the catheter 102.

The laser 124 of the catheter system 100 can be configured to provide one or more sub-millisecond energy pulses that are sent to and received by the optical fiber 122. The optical fiber 122 acts as a conduit for light energy that is generated by the energy pulse(s). In certain embodiments, the laser 124 can include one or more seed sources 126 and one or more amplifiers 128. Each amplifier 128 can be in optical communication with at least one of the seed sources 126. The seed source(s) 126 can each emit a relatively low-power seed pulse that is received and amplified by the amplifier 128. The amplifier 128 can increase the power of the seed pulse to generate the energy pulse. In one embodiment, the laser 124 can include one seed source 126 and one amplifier 128. Alternatively, the laser 124 can include a plurality of seed sources 126 and one amplifier 128. Still alternatively, the laser 124 can include a plurality of seed sources 126 and a plurality of amplifiers 128.

The light energy that is generated by the energy pulse(s) is delivered by the optical fiber 122 to a location within the balloon 104. The light energy induces plasma formation in the form of a plasma pulse 134 that occurs in the balloon fluid 132 within the balloon 104. The plasma pulse 134 causes rapid bubble formation and imparts pressure waves upon the treatment site 106. Exemplary plasma pulses 134 are shown in FIG. 1. The balloon fluid 132 can be a liquid or a gas. As provided in greater detail herein, the plasma-induced bubbles 134 are intentionally formed at some distance away from the optical fiber 122 so that the likelihood of damage to the optical fiber is decreased.

In various embodiments, the sub-millisecond pulses of light can be delivered to near the treatment site 106 at a frequency of from at least approximately 1 hertz (Hz) up to approximately 5000 Hz. In some embodiments, the sub-millisecond pulses of light can be delivered to near the treatment site 106 at a frequency from at least 30 Hz to 1000 Hz. In other embodiments, the sub-millisecond pulses of light can be delivered to near the treatment site 106 at a frequency from at least 10 Hz to 100 Hz. In yet other embodiments, the sub-millisecond pulses of light can be delivered to near the treatment site 106 at a frequency from at least 1 Hz to 30 Hz.

It is appreciated that the catheter system 100 herein can include any number of optical fibers 122 in optical communication with the laser 124 at the proximal portion 114, and with the balloon fluid 132 within the balloon 104 at the distal portion 116. For example, in some embodiments, the catheter system 100 herein can include 1-30 optical fibers 122. In some embodiments, the catheter system 100 herein can include greater than 30 optical fibers.

The manifold 136 can be positioned at or near the shaft proximal end 114. The manifold 136 can include one or more proximal end openings that can receive the one or more optical fibers, such as optical fiber 122, the guidewire 112, and/or an inflation conduit 140. The catheter system 100 can also include the fluid pump 138 that is configured to inflate the balloon 104 with the balloon fluid 132 and/or deflate the balloon 104 as needed.

As with all embodiments illustrated and described herein, various structures may be omitted from the figures for clarity and ease of understanding. Further, the figures may include certain structures that can be omitted without deviating from the intent and scope of the invention. Unless otherwise stated, all figures and descriptions refer to the balloon 104 in an inflated state 135. The balloon 104 can be movable between the inflated state and a deflated state (not shown).

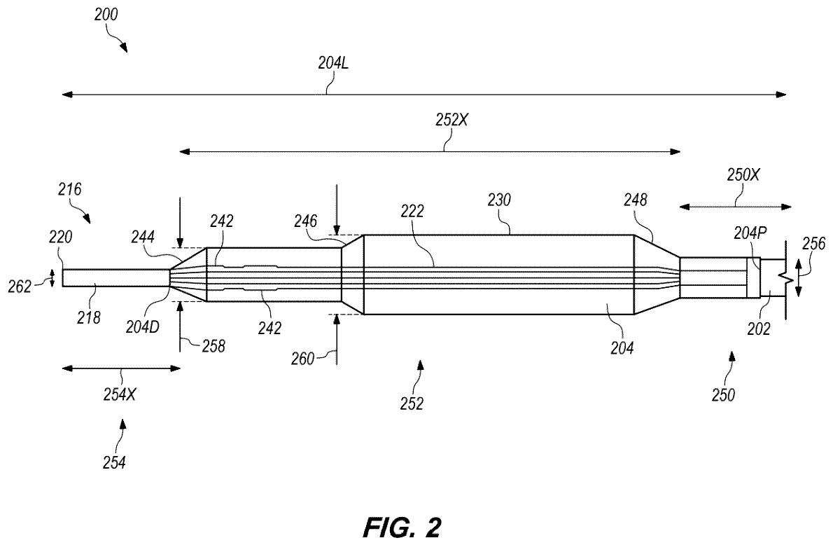

FIG. 2 is a partially transparent front view of one embodiment of a portion of the catheter system 200, including the optical fiber 122. It is appreciated that various components of the catheter system 200, such as are shown in FIG. 1, are not illustrated in FIG. 2 for purposes of clarity and ease of illustration. However, it is appreciated that the catheter system 200 can include most, if not all, of such components.

As shown in FIG. 2, the catheter system 200 can include a catheter 202 and an inflatable balloon 204. The inflatable balloon 204 can have a balloon wall 230, a balloon distal end 204D, and a balloon proximal end 204P. The catheter 202 can have a shaft distal end 216. The guidewire lumen 218 can extend through the shaft distal end 216, and the shaft distal end 216 can include a distal end opening 220. The optical fiber 222 can extend through and along a length of the catheter system 200. In other embodiments, the optical fiber 222 can begin at the fiber proximal end 122P and can end at the balloon distal end 204D. As used herein, the term “optical fiber” can refer equally to a bundle of optical fibers 222 or a single optical fiber 222.

In the embodiment illustrated in FIG. 2, the catheter system 200 can include one or more emitter(s) 242 distributed along an active length(s) of the calcified vascular lesion(s) located at the treatment site 106. The energy source described herein can be any suitable energy source for use within the catheter system 100. In some embodiments, the optical fiber 222 can be substituted with any suitable light carrier configured to receive an energy pulse. The optical fiber 222 can receive the energy pulse and can direct the energy pulse toward the emitter 242.

The emitter 242 can be driven by the energy source. The emitter 242 can be a plasma generator. The emitter 242 can be located at any position along the portion of the optical fiber 222 located inside the balloon 204. The emitter 242 can produce one or more plasma pulses 134.

The emitter 242 can vary depending on the design requirements of the catheter system 200, the optical fiber 222, and/or the laser 224 (or equivalent energy source). It is understood that the emitter 242 can include additional systems, subsystems, components, and elements than those specifically shown and/or described herein. Additionally, or alternatively, the emitter 242 can omit one or more of the systems, subsystems, and elements that are specifically shown and/or described herein.

As illustrated in the embodiment illustrated in FIG. 2, the balloon 204 can include transition regions that each have transition portions, transition region lengths, and transition region diameters. In one non-exclusive embodiment, the balloon 204 can include a first transition portion 244, a second transition portion 246, and a third transition portion 248.

The balloon 204 can have defined regions such as a balloon proximal region 250 having a balloon proximal region length 250X, a balloon transition region 252 having a balloon transition region length 252X, and a balloon distal region 254 having a balloon distal region length 254X. As used herein, a total length of the balloon proximal region length 250X plus the balloon transition region length 252X plus the balloon distal region length 254X equals the “balloon length” 204L. The balloon proximal region 250 can include a balloon proximal region diameter 256. The balloon transition region 252 can include any number of diameters, including a second transition region diameter 258 and a first transition region diameter 260. The balloon distal region 254 can include a balloon distal region diameter 262.

While FIG. 2 illustrates a number of lengths and portions, it is appreciated that the balloon 204 can include any number of defined lengths and portions. For example, the first transition portion 244 can have a first transition region length having an infinite number of diameters. The balloon 204 can have proximal and distal regions that have constant diameters throughout the region. In other words, the balloon 204 lengths in FIG. 2 are illustrative and can be demonstrative of any region of the balloon 204. The balloon 204 lengths can vary.

In various embodiments, the balloon 204 lengths (e.g., the balloon proximal region length 250X, the balloon transition region length 252X, and the balloon distal region length 254X) described herein can each have lengths of 0.5 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, 10.5 mm, 11 mm, 11.5 mm, 12 mm, 12.5 mm, 13 mm, 13.5 mm, 14 mm, 14.5 mm, 15 mm, 15.5 mm, 16 mm, 16.5 mm, 17 mm, 17.5 mm, 18 mm, 18.5 mm, 19 mm, 19.5 mm, 20 mm, 20.5 mm, 21 mm, 21.5 mm, 22 mm, 22.5 mm, 23 mm, 23.5 mm, 24 mm, 24.5 mm, 25 mm, 25.5 mm, 26 mm, 26.5 mm, 27 mm, 27.5 mm, 28 mm, 28.5 mm, 29 mm, 29.5 mm, 30 mm, 30.5 mm, 31 mm, 31.5 mm, 32 mm, 32.5 mm, 33 mm, 33.5 mm, 34 mm, 34.5 mm, 35 mm, 35.5 mm, 36 mm, 36.5 mm, 37 mm, 37.5 mm, 38 mm, 38.5 mm, 39 mm, 39.5 mm, 40 mm, 40.5 mm, 41 mm, 41.5 mm, 42 mm, 42.5 mm, 43 mm, 43.5 mm, 44 mm, 44.5 mm, 45 mm, 45.5 mm, 46 mm, 46.5 mm, 47 mm, 47.5 mm, 48 mm, 48.5 mm, 49 mm, 49.5 mm, 50 mm, 50.5 mm, 51 mm, 51.5 mm, 52 mm, 52.5 mm, 53 mm, 53.5 mm, 54 mm, 54.5 mm, 55 mm, 55.5 mm, 56 mm, 56.5 mm, 57 mm, 57.5 mm, 58 mm, 58.5 mm, 59 mm, 59.5 mm, 60 mm, 60.5 mm, 61 mm, 61.5 mm, 62 mm, 62.5 mm, 63 mm, 63.5 mm, 64 mm, 64.5 mm, 65 mm, 65.5 mm, 66 mm, 66.5 mm, 67 mm, 67.5 mm, 68 mm, 68.5 mm, 69 mm, 69.5 mm, 70 mm, 70.5 mm, 71 mm, 71.5 mm, 72 mm, 72.5 mm, 73 mm, 73.5 mm, 74 mm, 74.5 mm, 75 mm, 75.5 mm, 76 mm, 76.5 mm, 77 mm, 77.5 mm, 78 mm, 78.5 mm, 79 mm, 79.5 mm, 80 mm, 80.5 mm, 81 mm, 81.5 mm, 82 mm, 82.5 mm, 83 mm, 83.5 mm, 84 mm, 84.5 mm, 85 mm, 85.5 mm, 86 mm, 86.5 mm, 87 mm, 87.5 mm, 88 mm, 88.5 mm, 89 mm, 89.5 mm, 90 mm, 90.5 mm, 91 mm, 91.5 mm, 92 mm, 92.5 mm, 93 mm, 93.5 mm, 94 mm, 94.5 mm, 95 mm, 95.5 mm, 96 mm, 96.5 mm, 97 mm, 97.5 mm, 98 mm, 98.5 mm, 99 mm, 99.5 mm, 100 mm, 100.5 mm, 101 mm, 101.5 mm, 102 mm, 102.5 mm, 103 mm, 103.5 mm, 104 mm, 104.5 mm, 105 mm, 105.5 mm, 106 mm, 106.5 mm, 107 mm, 107.5 mm, 108 mm, 108.5 mm, 109 mm, 109.5 mm, 110 mm, 110.5 mm, 111 mm, 111.5 mm, 112 mm, 112.5 mm, 113 mm, 113.5 mm, 114 mm, 114.5 mm, 115 mm, 115.5 mm, 116 mm, 116.5 mm, 117 mm, 117.5 mm, 118 mm, 118.5 mm, 119 mm, 119.5 mm, 120 mm, 120.5 mm, 121 mm, 121.5 mm, 122 mm, 122.5 mm, 123 mm, 123.5 mm, 124 mm, 124.5 mm, 125 mm, 125.5 mm, 126 mm, 126.5 mm, 127 mm, 127.5 mm, 128 mm, 128.5 mm, 129 mm, 129.5 mm, 130 mm, 130.5 mm, 131 mm, 131.5 mm, 132 mm, 132.5 mm, 133 mm, 133.5 mm, 134 mm, 134.5 mm, 135 mm, 135.5 mm, 136 mm, 136.5 mm, 137 mm, 137.5 mm, 138 mm, 138.5 mm, 139 mm, 139.5 mm, 140 mm, 140.5 mm, 141 mm, 141.5 mm, 142 mm, 142.5 mm, 143 mm, 143.5 mm, 144 mm, 144.5 mm, 145 mm, 145.5 mm, 146 mm, 146.5 mm, 147 mm, 147.5 mm, 148 mm, 148.5 mm, 149 mm, 149.5 mm, 150 mm, 150.5 mm, 151 mm, 151.5 mm, 152 mm, 152.5 mm, 153 mm, 153.5 mm, 154 mm, 154.5 mm, 155 mm, 155.5 mm, 156 mm, 156.5 mm, 157 mm, 157.5 mm, 158 mm, 158.5 mm, 159 mm, 159.5 mm, 160 mm, 160.5 mm, 161 mm, 161.5 mm, 162 mm, 162.5 mm, 163 mm, 163.5 mm, 164 mm, 164.5 mm, 165 mm, 165.5 mm, 166 mm, 166.5 mm, 167 mm, 167.5 mm, 168 mm, 168.5 mm, 169 mm, 169.5 mm, 170 mm, 170.5 mm, 171 mm, 171.5 mm, 172 mm, 172.5 mm, 173 mm, 173.5 mm, 174 mm, 174.5 mm, 175 mm, 175.5 mm, 176 mm, 176.5 mm, 177 mm, 177.5 mm, 178 mm, 178.5 mm, 179 mm, 179.5 mm, 180 mm, 180.5 mm, 181 mm, 181.5 mm, 182 mm, 182.5 mm, 183 mm, 183.5 mm, 184 mm, 184.5 mm, 185 mm, 185.5 mm, 186 mm, 186.5 mm, 187 mm, 187.5 mm, 188 mm, 188.5 mm, 189 mm, 189.5 mm, 190 mm, 190.5 mm, 191 mm, 191.5 mm, 192 mm, 192.5 mm, 193 mm, 193.5 mm, 194 mm, 194.5 mm, 195 mm, 195.5 mm, 196 mm, 196.5 mm, 197 mm, 197.5 mm, 198 mm, 198.5 mm, 199 mm, 199.5 mm, 200 mm, 200.5 mm, 201 mm, 201.5 mm, 202 mm, 202.5 mm, 203 mm, 203.5 mm, 204 mm, 204.5 mm, 205 mm, 205.5 mm, 206 mm, 206.5 mm, 207 mm, 207.5 mm, 208 mm, 208.5 mm, 209 mm, 209.5 mm, 210 mm, 210.5 mm, 211 mm, 211.5 mm, 212 mm, 212.5 mm, 213 mm, 213.5 mm, 214 mm, 214.5 mm, 215 mm, 215.5 mm, 216 mm, 216.5 mm, 217 mm, 217.5 mm, 218 mm, 218.5 mm, 219 mm, 219.5 mm, 220 mm, 220.5 mm, 221 mm, 221.5 mm, 222 mm, 222.5 mm, 223 mm, 223.5 mm, 224 mm, 224.5 mm, 225 mm, 225.5 mm, 226 mm, 226.5 mm, 227 mm, 227.5 mm, 228 mm, 228.5 mm, 229 mm, 229.5 mm, 230 mm, 230.5 mm, 231 mm, 231.5 mm, 232 mm, 232.5 mm, 233 mm, 233.5 mm, 234 mm, 234.5 mm, 235 mm, 235.5 mm, 236 mm, 236.5 mm, 237 mm, 237.5 mm, 238 mm, 238.5 mm, 239 mm, 239.5 mm, 240 mm, 240.5 mm, 241 mm, 241.5 mm, 242 mm, 242.5 mm, 243 mm, 243.5 mm, 244 mm, 244.5 mm, 245 mm, 245.5 mm, 246 mm, 246.5 mm, 247 mm, 247.5 mm, 248 mm, 248.5 mm, 249 mm, 249.5 mm, 250 mm, 250.5 mm, 251 mm, 251.5 mm, 252 mm, 252.5 mm, 253 mm, 253.5 mm, 254 mm, 254.5 mm, 255 mm, 255.5 mm, 256 mm, 256.5 mm, 257 mm, 257.5 mm, 258 mm, 258.5 mm, 259 mm, 259.5 mm, 260 mm, 260.5 mm, 261 mm, 261.5 mm, 262 mm, 262.5 mm, 263 mm, 263.5 mm, 264 mm, 264.5 mm, 265 mm, 265.5 mm, 266 mm, 266.5 mm, 267 mm, 267.5 mm, 268 mm, 268.5 mm, 269 mm, 269.5 mm, 270 mm, 270.5 mm, 271 mm, 271.5 mm, 272 mm, 272.5 mm, 273 mm, 273.5 mm, 274 mm, 274.5 mm, 275 mm, 275.5 mm, 276 mm, 276.5 mm, 277 mm, 277.5 mm, 278 mm, 278.5 mm, 279 mm, 279.5 mm, 280 mm, 280.5 mm, 281 mm, 281.5 mm, 282 mm, 282.5 mm, 283 mm, 283.5 mm, 284 mm, 284.5 mm, 285 mm, 285.5 mm, 286 mm, 286.5 mm, 287 mm, 287.5 mm, 288 mm, 288.5 mm, 289 mm, 289.5 mm, 290 mm, 290.5 mm, 291 mm, 291.5 mm, 292 mm, 292.5 mm, 293 mm, 293.5 mm, 294 mm, 294.5 mm, 295 mm, 295.5 mm, 296 mm, 296.5 mm, 297 mm, 297.5 mm, 298 mm, 298.5 mm, 299 mm, 299.5 mm, 300 mm. It is appreciated that the balloon 204 lengths illustrated and/or described herein have lengths that can fall within a range, wherein any of the foregoing numbers can serve as the lower or upper bound of the range, provided that the lower bound of the range is a value less than the upper bound of the range. The balloon 204 lengths described herein can have lengths that fall outside of the range described herein.

In various embodiments, the balloon 204 diameters (e.g, the balloon proximal region diameter, the balloon transition region diameter, and the balloon distal region diameter) described herein can each have diameters of 0.1 mm, 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, 5 mm, 5.1 mm, 5.2 mm, 5.3 mm, 5.4 mm, 5.5 mm, 5.6 mm, 5.7 mm, 5.8 mm, 5.9 mm, 6 mm, 6.1 mm, 6.2 mm, 6.3 mm, 6.4 mm, 6.5 mm, 6.6 mm, 6.7 mm, 6.8 mm, 6.9 mm, 7 mm, 7.1 mm, 7.2 mm, 7.3 mm, 7.4 mm, 7.5 mm, 7.6 mm, 7.7 mm, 7.8 mm, 7.9 mm, 8 mm, 8.1 mm, 8.2 mm, 8.3 mm, 8.4 mm, 8.5 mm, 8.6 mm, 8.7 mm, 8.8 mm, 8.9 mm, 9 mm, 9.1 mm, 9.2 mm, 9.3 mm, 9.4 mm, 9.5 mm, 9.6 mm, 9.7 mm, 9.8 mm, 9.9 mm, 10 mm, 10.1 mm, 10.2 mm, 10.3 mm, 10.4 mm, 10.5 mm, 10.6 mm, 10.7 mm, 10.8 mm, 10.9 mm, 11 mm, 11.1 mm, 11.2 mm, 11.3 mm, 11.4 mm, 11.5 mm, 11.6 mm, 11.7 mm, 11.8 mm, 11.9 mm, 12 mm, 12.1 mm, 12.2 mm, 12.3 mm, 12.4 mm, 12.5 mm, 12.6 mm, 12.7 mm, 12.8 mm, 12.9 mm, 13 mm, 13.1 mm, 13.2 mm, 13.3 mm, 13.4 mm, 13.5 mm, 13.6 mm, 13.7 mm, 13.8 mm, 13.9 mm, 14 mm, 14.1 mm, 14.2 mm, 14.3 mm, 14.4 mm, 14.5 mm, 14.6 mm, 14.7 mm, 14.8 mm, 14.9 mm, 15 mm, 15.1 mm, 15.2 mm, 15.3 mm, 15.4 mm, 15.5 mm, 15.6 mm, 15.7 mm, 15.8 mm, 15.9 mm, 16 mm, 16.1 mm, 16.2 mm, 16.3 mm, 16.4 mm, 16.5 mm, 16.6 mm, 16.7 mm, 16.8 mm, 16.9 mm, 17 mm, 17.1 mm, 17.2 mm, 17.3 mm, 17.4 mm, 17.5 mm, 17.6 mm, 17.7 mm, 17.8 mm, 17.9 mm, 18 mm, 18.1 mm, 18.2 mm, 18.3 mm, 18.4 mm, 18.5 mm, 18.6 mm, 18.7 mm, 18.8 mm, 18.9 mm, 19 mm, 19.1 mm, 19.2 mm, 19.3 mm, 19.4 mm, 19.5 mm, 19.6 mm, 19.7 mm, 19.8 mm, 19.9 mm, 20 mm, 20.1 mm, 20.2 mm, 20.3 mm, 20.4 mm, 20.5 mm, 20.6 mm, 20.7 mm, 20.8 mm, 20.9 mm, 21 mm, 21.1 mm, 21.2 mm, 21.3 mm, 21.4 mm, 21.5 mm, 21.6 mm, 21.7 mm, 21.8 mm, 21.9 mm, 22 mm, 22.1 mm, 22.2 mm, 22.3 mm, 22.4 mm, 22.5 mm, 22.6 mm, 22.7 mm, 22.8 mm, 22.9 mm, 23 mm, 23.1 mm, 23.2 mm, 23.3 mm, 23.4 mm, 23.5 mm, 23.6 mm, 23.7 mm, 23.8 mm, 23.9 mm, 24 mm, 24.1 mm, 24.2 mm, 24.3 mm, 24.4 mm, 24.5 mm, 24.6 mm, 24.7 mm, 24.8 mm, 24.9 mm, 25 mm, 25.1 mm, 25.2 mm, 25.3 mm, 25.4 mm, 25.5 mm, 25.6 mm, 25.7 mm, 25.8 mm, 25.9 mm, 26 mm, 26.1 mm, 26.2 mm, 26.3 mm, 26.4 mm, 26.5 mm, 26.6 mm, 26.7 mm, 26.8 mm, 26.9 mm, 27 mm, 27.1 mm, 27.2 mm, 27.3 mm, 27.4 mm, 27.5 mm, 27.6 mm, 27.7 mm, 27.8 mm, 27.9 mm, 28 mm, 28.1 mm, 28.2 mm, 28.3 mm, 28.4 mm, 28.5 mm, 28.6 mm, 28.7 mm, 28.8 mm, 28.9 mm, 29 mm, 29.1 mm, 29.2 mm, 29.3 mm, 29.4 mm, 29.5 mm, 29.6 mm, 29.7 mm, 29.8 mm, 29.9 mm, 30 mm. It is appreciated that the balloon 204 diameters illustrated and/or described herein have diameters that can fall within a range, wherein any of the foregoing numbers can serve as the lower or upper bound of the range, provided that the lower bound of the range is a value less than the upper bound of the range. The balloon 204 diameters described herein can have lengths that fall outside of the range described herein.

The first transition portion 244 can transition the balloon distal region diameter 262 from the balloon distal end 204D to the first transition region diameter 260. The first transition portion 244 can vary depending on the design requirements of the catheter system 200, the optical fiber 222, and/or the emitter 242 (or equivalent). It is understood that the first transition portion 244 can include additional systems, subsystems, components, and elements than those specifically shown and/or described herein. Additionally, or alternatively, the first transition portion 244 can omit one or more of the systems, subsystems, and elements that are specifically shown and/or described herein. The first transition portion 244 can have any suitable design, including but not limited to, curves, lines, angles, etc. It is appreciated that the balloon 204 can include any number of transition portions necessary for any number of corresponding transition regions.

The second transition portion 246 can be substantially similar to the first transition portion 244. The second transition portion 246 can transition the first transition region diameter 260 to the second transition region diameter 258.

The third transition portion 248 can be substantially similar to the first transition portion 244. The third transition portion 248 can transition the second transition region diameter 258 to the balloon proximal region diameter 256.

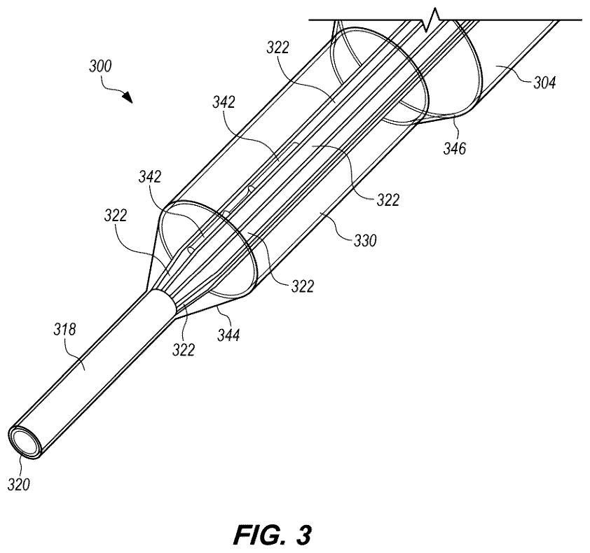

FIG. 3 is a partially transparent perspective view of the embodiment of a portion of the catheter system 300 shown in FIG. 2 including the optical fibers 322. The catheter system 300 can be substantially similar to the other catheter systems described herein.

As shown in the embodiment illustrated in FIG. 3, the catheter system 300 can include two emitters 342 spaced apart to target the treatment site 106. The number of emitters 342 and spacing between the emitter 342 can vary depending on the treatment site 106 location and size of the lesion, blood vessel, and/or other targets within the treatment site 106.

In some embodiments, the balloon 304 can be formed around the guidewire lumen 318, and the balloon 304 can extend to the distal end opening 320. In other embodiments (such as those previously described herein), the balloon 304 can only be formed around the optical fibers 322 and can end at the balloon distal end 204D, instead of the distal end opening 320.

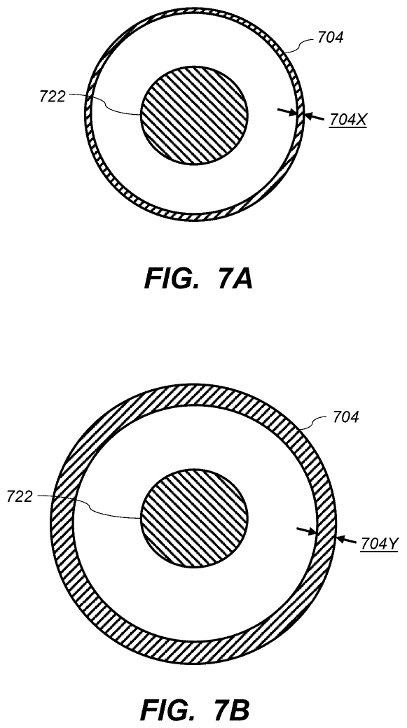

The balloon wall 330 can have a thickness (e.g., a first wall thickness 704X and/or a second wall thickness 704Y, as illustrated in FIGS. 7A-7B) and can be somewhat linear and/or cylindrical in areas with a constant diameter (e.g., not the first transition portion 344 and the second transition portion 346). In some embodiments, the balloon wall 330 thickness can vary, as described herein.

The first transition portion 344 and the second transition portion 346 can each be somewhat conical, but they can also be cylindrical, tapered, jagged, or have a shape that is somewhat similar to letters of the English alphabet (e.g., U-shaped, M-shaped, V-shaped, etc.).

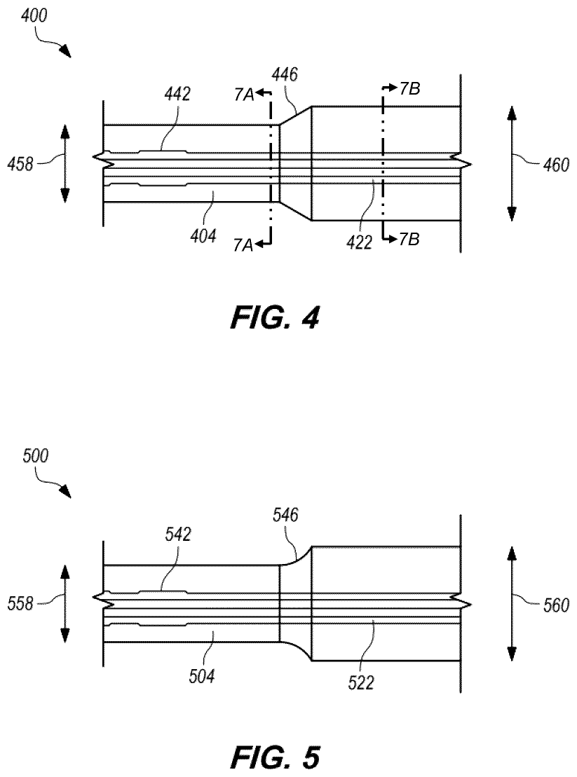

FIG. 4 is a partially transparent front view of one embodiment of a portion of the catheter system 400 including an optical fiber 422. The catheter system 400 can be substantially similar to the other catheter systems described herein. In some embodiments, the balloon 404 includes only one transition portion (such as the first transition portion 244 or the second transition portion 446), so that there is only one transition in diameters of the balloon 404. As used herein, the first transition portion and/or the second transition portion can be referred to simply as a“transition portion.”

As shown in the embodiment illustrated in FIG. 4, the catheter system 400 can include a linear second transition portion 446 that can transition the diameter of the balloon 404 from the first transition region diameter 458 to the second transition region diameter 460. In other embodiments, the second transition portion 446 can transition the diameter of the balloon 404 from the second transition region diameter 460 to the first transition region diameter 458. FIG. 4 also includes cross-sectional lines 7A-7A and 7B-7B for cross-sectional views shown in FIGS. 7A and 7B, respectively.

FIG. 5 is a partially transparent front view of one embodiment of a portion of the catheter system 500 including an optical fiber 522. The catheter system 500 can be substantially similar to the other catheter systems described herein.

As shown in the embodiment illustrated in FIG. 5, the catheter system 500 can include a curved (e.g., non-linear and/or angular) second transition portion 546 that can transition the diameter of the balloon 504 from the first transition region diameter 558 to the second transition region diameter 560. In other embodiments, the second transition portion 546 can transition the diameter of the balloon 504 from the second transition region diameter 560 to the first transition region diameter 558. As used herein, the first transition portion and/or the second transition portion can be referred to simply as a“transition portion.”

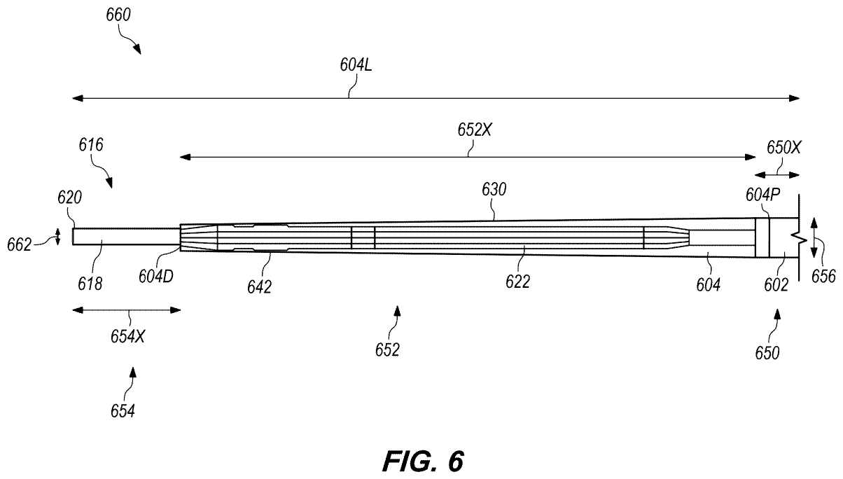

FIG. 6 is a partially transparent front view of one embodiment of a portion of the catheter system 600 including an optical fiber 622. With various exceptions, the catheter system 600 can be substantially similar to the other catheter systems described herein.

In the embodiment illustrated in FIG. 6, the balloon 604 has a somewhat conical design. Similar to previous embodiments, the catheter system 600 can include a catheter 602 and the balloon 604 having a balloon distal end 604D, a balloon proximal end 604P, and a balloon wall 630. The guidewire lumen 618 can extend through the shaft distal end 616, and the guidewire lumen 618 can have a distal end opening 620 for the guidewire 112. The optical fiber 622 can include one or more emitters 642.

The balloon 604 can have defined regions such as a balloon proximal region 650 having a balloon proximal region length 650X, a balloon transition region 652 having a balloon transition region length 652X, and a balloon distal region 654 having a balloon distal region length 654X. As used herein, a total length of the balloon proximal region length 650X plus the balloon transition region length 652X plus the balloon distal region length 654X equals the “balloon length” 604L. The balloon proximal region 650 can include a balloon proximal region diameter 656. In various embodiments, the balloon transition region 652 can include two or more different diameters (such as an infinite number of diameters) and can include a second transition region diameter 658 and a first transition region diameter 660. The balloon distal region 654 can include a balloon distal region diameter 662.

FIG. 7A is a simplified cross-sectional view of one embodiment of a portion of the catheter system 400 (illustrated in FIG. 4) taken on line 7A-7A in FIG. 4. As shown in FIGS. 7A and 7B, the balloon 704 and the balloon wall 230 (illustrated in FIG. 2) can have varying wall thicknesses along the balloon length 204L (illustrated in FIG. 2). A first wall thickness 704X is illustrated in FIG. 7A, and a second wall thickness 704Y is illustrated in FIG. 7B. The wall thicknesses 704X and 704Y can also vary depending on the design requirements of the optical fiber 722.

The wall thicknesses shown in FIG. 7A and FIG. 7B are merely representative, and it is understood that the first wall thickness 704X can be greater than, less than, or equal to the second wall thickness 704Y. Also, the use of “first” and “second” wall thicknesses 704X, 704Y are for ease of understanding, and it is understood that the “first” wall thickness and the “second” wall thickness may be used interchangeably in that the “first” wall thickness may be the “second” wall thickness in some embodiments, and vice versa. It is also understood that the balloon 704 can include any suitable number of wall thicknesses (e.g., an infinite number of wall thicknesses) in order to meet its design requirements. It is also understood that the wall thicknesses 704X, 704Y can represent any suitable wall thickness on any portion of the inflatable balloon 704 along the entirety of the balloon length 204L.

In certain embodiments, the balloon distal region 254 (illustrated in FIG. 2) can include the first wall thickness 704X, and the balloon proximal region 250 (illustrated in FIG. 2) can include the second wall thickness 704Y. In some such embodiments, the second wall thickness 704Y is greater than the first wall thickness 704X.

In some embodiments, the balloon proximal region 250 (illustrated in FIG. 2) can include the first wall thickness 704X, and the balloon distal region 254 (illustrated in FIG. 2) can include the second wall thickness 704Y. In some such embodiments, the second wall thickness 704Y is greater than the first wall thickness 704X.

In various embodiments, the balloon distal region 254 (illustrated in FIG. 2) can include the first wall thickness 704X, and the balloon transition region 252 (illustrated in FIG. 2) can include the second wall thickness 704Y. In some such embodiments, the second wall thickness 704Y is greater than the first wall thickness 704X.

In certain embodiments, the balloon transition region 252 (illustrated in FIG. 2) can include the first wall thickness 704X, and the balloon distal region 254 (illustrated in FIG. 2) can include the second wall thickness 704Y. In some such embodiments, the second wall thickness 704Y is greater than the first wall thickness 704X.

In some embodiments, the balloon proximal region 250 (illustrated in FIG. 2) can include the first wall thickness 704X, and the balloon transition region 252 (illustrated in FIG. 2) can include the second wall thickness 704Y. In some such embodiments, the second wall thickness 704Y is greater than the first wall thickness 704X.

In various embodiments, the balloon transition region 252 (illustrated in FIG. 2) can include the first wall thickness 704X, and the balloon proximal region 250 (illustrated in FIG. 2) can include the second wall thickness 704Y. In some such embodiments, the second wall thickness 704Y is greater than the first wall thickness 704X.

In some embodiments, a ratio of the first wall thickness 704X to the second wall thickness 704Y for the balloon 704 herein can be between approximately 1:10 and 10:1. In some such non-exclusive embodiments, the ratio of the first wall thickness 704X to the second wall thickness 704Y for the balloon 704 can be approximately 1:10, 1:9.9, 1:9.8, 1:9.7, 1:9.6, 1:9.5, 1:9.4, 1:9.3, 1:9.2, 1:9.1, 1:9, 1:8.9, 1:8.8, 1:8.7, 1:8.6, 1:8.5, 1:8.4, 1:8.3, 1:8.2, 1:8.1, 1:8, 1:7.9, 1:7.8, 1:7.7, 1:7.6, 1:7.5, 1:7.4, 1:7.3, 1:7.2, 1:7.1, 1:7, 1:6.9, 1:6.8, 1:6.7, 1:6.6, 1:6.5, 1:6.4, 1:6.3, 1:6.2, 1:6.1, 1:6, 1:5.9, 1:5.8, 1:5.7, 1:5.6, 1:5.5, 1:5.4, 1:5.3, 1:5.2, 1:5.1, 1:5, 1:4.9, 1:4.8, 1:4.7, 1:4.6, 1:4.5, 1:4.4, 1:4.3, 1:4.2, 1:4.1, 1:4, 1:3.9, 1:3.8, 1:3.7, 1:3.6, 1:3.5, 1:3.4, 1:3.3, 1:3.2, 1:3.1, 1:3, 1:2.9, 1:2.8, 1:2.7, 1:2.6, 1:2.5, 1:2.4, 1:2.3, 1:2.2, 1:2.1, 1:2, 1:1.9, 1:1.8, 1:1.7, 1:1.6, 1:1.5, 1:1.4, 1:1.3, 1:1.2, 1:1.1, 1:1, 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, 5.1:1, 5.2:1, 5.3:1, 5.4:1, 5.5:1, 5.6:1, 5.7:1, 5.8:1, 5.9:1, 6:1, 6.1:1, 6.2:1, 6.3:1, 6.4:1, 6.5:1, 6.6:1, 6.7:1, 6.8:1, 6.9:1, 7:1, 7.1:1, 7.2:1, 7.3:1, 7.4:1, 7.5:1, 7.6:1, 7.7:1, 7.8:1, 7.9:1, 8:1, 8.1:1, 8.2:1, 8.3:1, 8.4:1, 8.5:1, 8.6:1, 8.7:1, 8.8:1, 8.9:1, 9:1, 9.1:1, 9.2:1, 9.3:1, 9.4:1, 9.5:1, 9.6:1, 9.7:1, 9.8:1, 9.9:1, or 10:1. Alternatively, in some embodiments, the ratio of the first wall thickness 704X to the second wall thickness 704Y for the balloon 704 can be greater than approximately 10:1 or less than approximately 1:10.

### Balloons

The balloons suitable for use herein can be coupled to various portions of the catheter system, including but not limited to, the catheter shaft. In some instances, the balloon may include dimensionalities such as balloon diameters, balloon waists, and balloon regions. An inflation lumen can be in fluid communication with the balloon.

In some instances, the balloon may be structurally reinforced. For example, the balloon may include a reinforcing member. The reinforcing member may be a layer of material disposed along the inner surface of the balloon, the outer surface of the balloon, or both. In certain embodiments, the reinforcing member may include a braid (e.g., a fiber braid), coil, or the like. In some of these and in other instances, the reinforcing member may be disposed along the inner surface of the balloon, the outer surface of the balloon, or be embedded between two layers of the balloon. The reinforcing member may provide additional structural support to the balloon, which may increase the burst strength of the balloon. In addition, the reinforcing member may fortify the balloon.

When suitably positioned, the balloon can be partially inflated and/or fully inflated. Partially inflating the balloon may occur by infusing an inflation media into the balloon (e.g., via an inflation lumen). Partially inflating the balloon may include inflating the balloon so that the balloon comes into contact with the wall of the blood vessel. This may include contacting the vessel wall or, in some instances, partially inflating the balloon may include partially expanding the blood vessel. In some instances, partially inflating the balloon may include inflating the balloon to a first pressure that might be in the range of about 1-6 atmospheres or about 3-5 atmospheres.

### Lasers

The lasers suitable for use herein can include various types of lasers, including lasers and lamps. Suitable lasers can include short pulse lasers on the sub-millisecond timescale. In some embodiments, the laser can include lasers on the nanosecond (ns) timescale. The lasers can also include short pulse lasers on the picosecond (ps), femtosecond (fs), and microsecond (us) timescales. It is appreciated that there are many combinations of laser wavelengths, pulse widths, and energy levels that can be employed to achieve plasma in the balloon fluid of the catheters illustrated and/or described herein. In various embodiments, the pulse widths can include those falling within a range, including from at least 10 ns to 200 ns. In some embodiments, the pulse widths can include those falling within a range, including from at least 20 ns to 100 ns. In other embodiments, the pulse widths can include those falling within a range, including from at least 1 ns to 5000 ns.

Exemplary nanosecond lasers can include those within the UV to IR spectrum, spanning wavelengths of about 10 nanometers to 1 millimeter. In some embodiments, the lasers suitable for use in the catheter systems herein can include those capable of producing light at wavelengths of from at least 350 nm to 2000 nm. In some embodiments, the lasers can include those capable of producing light at wavelengths of from at least 700 nm to 3000 nm. In some embodiments, the lasers can include those capable of producing light at wavelengths of from at least 100 nm to 10 micrometers (mm). Nanosecond lasers can include those having repetition rates of up to 200 kHz. In some embodiments, the laser can include a Q-switched thulium:yttrium-aluminum-garnet (Tm:YAG) laser. In some embodiments, the laser can include a neodymium:yttrium-aluminum-garnet (Nd:YAG), holmium:yttrium-aluminum-garnet (Ho:YAG), erbium:yttrium-aluminum-garnet (Er:YAG), excimer laser, helium-neon laser, carbon dioxide laser, as well as doped, pulsed, fiber lasers.

### Pressure Waves

The catheters illustrated and/or described herein can generate pressure waves having maximum pressures in the range of at least 1 megapascal (MPa) to 100 MPa. The maximum pressure generated by a particular catheter will depend on the laser, the absorbing material, the bubble expansion, the propagation medium, the balloon material, and other factors. In some embodiments, the catheters illustrated and/or described herein can generate pressure waves having maximum pressures in the range of at least 2 MPa to 50 MPa. In other embodiments, the catheters illustrated and/or described herein can generate pressure waves having maximum pressures in the range of at least 2 MPa to 30 MPa. In yet other embodiments, the catheters illustrated and/or described herein can generate pressure waves having maximum pressures in the range of at least 15 MPa to 25 MPa. In some embodiments, the catheters illustrated and/or described herein can generate pressure waves having peak pressures of greater than or equal to 1 MPa, 2 MPa, 3 MPa, 4 MPa, 5 MPa, 6 MPa, 7 MPa, 8 MPa, 9 MPa, 10 MPa, 11 MPa, 12 MPa, 13 MPa, 14 MPa, 15 MPa, 16 MPa, 17 MPa, 18 MPa, 19 MPa, 20 MPa, 21 MPa, 22 MPa, 23 MPa, 24 MPa, or 25 MPa, 26 MPa, 27 MPa, 28 MPa, 29 MPa, 30 MPa, 31 MPa, 32 MPa, 33 MPa, 34 MPa, 35 MPa, 36 MPa, 37 MPa, 38 MPa, 39 MPa, 40 MPa, 41 MPa, 42 MPa, 43 MPa, 44 MPa, 45 MPa, 46 MPa, 47 MPa, 48 MPa, 49 MPa, or 50 MPa. It is appreciated that the catheters illustrated and/or described herein can generate pressure waves having operating pressures or maximum pressures that can fall within a range, wherein any of the foregoing numbers can serve as the lower or upper bound of the range, provided that the lower bound of the range is a value less than the upper bound of the range.

Therapeutic treatment can act via a fatigue mechanism or a brute force mechanism. For a fatigue mechanism, operating pressures would be about at least 0.5 MPa to 2 MPa, or about 1 MPa. For a brute force mechanism, operating pressures would be about at least 20 MPa to 30 MPa, or about 25 MPa. Pressures between the extreme ends of these two ranges may act upon a treatment site using a combination of a fatigue mechanism and a brute force mechanism.

The pressure waves described herein can be imparted upon the treatment site from a distance within a range from at least 0.01 millimeters (mm) to 25 mm, extending radially from a longitudinal axis of a catheter placed at a treatment site. In some embodiments, the pressure waves can be imparted upon the treatment site from a distance within a range from at least 1 mm to 20 mm, extending radially from a longitudinal axis of a catheter placed at a treatment site. In other embodiments, the pressure waves can be imparted upon the treatment site from a distance within a range from at least 0.1 mm to 10 mm, extending radially from a longitudinal axis of a catheter placed at a treatment site. In yet other embodiments, the pressure waves can be imparted upon the treatment site from a distance within a range from at least 1.5 mm to 4 mm, extending radially from a longitudinal axis of a catheter placed at a treatment site. In some embodiments, the pressure waves can be imparted upon the treatment site from a range of at least 2 MPa to 30 MPa at a distance from 0.1 mm to 10 mm. In some embodiments, the pressure waves can be imparted upon the treatment site from a range of at least 2 MPa to 25 MPa at a distance from 0.1 mm to 10 mm. In some embodiments, the pressure waves can be imparted upon the treatment site from a distance that can be greater than or equal to 0.1 mm, 0.2 mm, 0.3 mm, 0.4 mm, 0.5 mm, 0.6 mm, 0.7 mm, 0.8 mm, or 0.9 mm, 1 mm, 2 mm, 3 mm, 4 mm, 5 mm, 6 mm, 7 mm, 8 mm, 9 mm, or 10 mm, or can be an amount falling within a range between, or outside the range of any of the foregoing.

The systems and methods described herein can provide a balloon for use with an intravascular lithotripsy catheter. A balloon having at least two different diameters can allow disruption of calcification in vessels with relatively small openings, and further allow enlargement of the vessel opening using a single intravascular lithotripsy catheter. In certain embodiments, a portion of the balloon with the smaller diameter can be advanced into a vessel, and an energy source can be initiated to perform intravascular lithotripsy therapy in order to allow further advancement of a portion of the balloon with a larger diameter. Stated another way, after intravascular lithotripsy is performed on an area of the vessel with less clearance, the catheter can be advanced forward to position the larger diameter portion in same area of the vessel to enlarge the vessel and complete the treatment.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

As used herein, the recitation of numerical ranges by endpoints shall include all numbers subsumed within that range, inclusive (e.g., 2 to 8 includes 2, 2.1, 2.8, 5.3, 7, 8, etc.).

It is recognized that the figures shown and described are not necessarily drawn to scale, and that they are provided for ease of reference and understanding, and for relative positioning of the structures.

The headings used herein are provided for consistency with suggestions under 37 CFR 1.77 or otherwise to provide organizational cues. These headings shall not be viewed to limit or characterize the invention(s) set out in any claims that may issue from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the catheter systems have been illustrated and described herein, one or more features of any one embodiment can be combined with one or more features of one or more of the other embodiments, provided that such combination satisfies the intent of the present invention.

While a number of exemplary aspects and embodiments of the catheter systems have been discussed above, those of skill in the art will recognize certain modifications, permutations, additions, and sub-combinations thereof. It is therefore intended that the following appended claims and claims hereafter introduced are interpreted to include all such modifications, permutations, additions, and sub-combinations as are within their true spirit and scope, and no limitations are intended to the details of construction or design herein shown.

What is claimed is:

1. A catheter system for treating a treatment site, comprising:
a balloon having a proximal end region, a distal end region and a body extending between the proximal end region and the distal end region; and
an optical fiber positioned within an inner cavity of the balloon, the optical fiber configured to receive light energy;
wherein the balloon body includes a first body portion having a first body portion outer diameter;
wherein the balloon body includes a second body portion having a second body portion outer diameter different from the first body portion outer diameter;
wherein the balloon body includes a first transition region positioned between the first body portion and the second body portion;
wherein the first transition region includes a first transition wall thickness and a first transition length, and wherein the first transition wall thickness varies along the first transition length.

2. The catheter system of claim 1, wherein the first transition region is linear along the first transition length.

3. The catheter system of claim 1, wherein the first transition region is curved along the first transition length.

4. The catheter system of claim 1, wherein an outer diameter of the first transition region increases from the first body portion to the second body portion.

5. The catheter system of claim 4, wherein the first transition wall thickness decreases along the first transition length from the first body portion to the second body portion.

6. The catheter system of claim 5, wherein the first body portion outer diameter is constant along the first body portion.

7. The catheter system of claim 6, wherein the second body portion outer diameter is constant along the second body portion.

8. The catheter system of claim 7, wherein the first body portion includes a first body portion wall thickness, and wherein the second body portion includes a second body portion wall thickness different from the first body portion wall thickness.

9. The catheter system of claim 8, wherein the second body portion wall thickness is thicker than the first body portion wall thickness.

10. The catheter system of claim 9, wherein the first body portion is positioned distal to the second body portion.

11. The catheter system of claim 1, wherein the distal end region includes a distal waist, and wherein the balloon further includes a second transition region positioned between the distal waist and the first body portion.

12. The catheter system of claim 11, wherein the proximal end region includes a proximal waist, and wherein the balloon further includes a third transition region positioned between the proximal waist and the second body portion.

13. The catheter system of claim 12, wherein the distal waist includes a distal waist outer diameter different from the first body portion outer diameter, the second body portion outer diameter, or both the first body portion outer diameter and the second body portion outer diameter.

14. The catheter system of claim 13, wherein the proximal waist includes a proximal waist outer diameter different from the first body portion outer diameter, the second body portion outer diameter and the distal waist outer diameter.

15. The catheter system of claim 1, wherein the balloon is configured to shift between an inflated configuration and a deflated configuration.

16. A catheter system for treating a treatment site, comprising:
a balloon having a proximal end region, a distal end region and a body extending between the proximal end region and the distal end region; and
an optical fiber positioned within an inner cavity of the balloon, the optical fiber configured to receive light energy;
wherein the balloon body includes a curved transition region positioned between the proximal end region and the distal end region.

17. The catheter system of claim 16, wherein the curved transition region includes a curved transition wall thickness and a curved transition length, and wherein the curved transition wall thickness varies along the curved transition length.

18. The catheter system of claim 17, wherein the balloon body includes a first body portion having a first body portion outer diameter, wherein the balloon body includes a second body portion having a second body portion outer diameter different from the first body portion outer diameter and wherein the curved transition region is positioned between the first body portion and the second body portion.

19. The catheter system of claim 18, wherein the distal end region includes a distal waist, wherein the balloon further includes a second transition region positioned between the distal waist and the first body portion, wherein the proximal end region includes a proximal waist and wherein the balloon further includes a third transition region positioned between the proximal waist and the second body portion.

20. A catheter system for treating a treatment site, comprising:
a balloon having a proximal end region, a distal end region and a body extending between the proximal end region and the distal end region; and
an optical fiber positioned within an inner cavity of the balloon, the optical fiber configured to receive light energy;
wherein the balloon body includes a first body portion;
wherein the balloon body includes a second body portion;
wherein the balloon body includes a first transition region positioned between the first body portion and the second body portion;
wherein the first transition region includes a first transition wall thickness and a first transition length, and wherein the first transition wall thickness tapers along the first transition length from the first body portion to the second body portion.
